(19) United States
(12) Patent Application Publication
(10) Pub. No.: US 2020/0231915 A1
(43) Pub. Date: Jul. 23, 2020

(54) PREPARATION METHOD AND APPLICATION OF COMPOUND MILK-BEER LYOPHILIZED FERMENTATION MICROORGANISM AGENT

(71) Applicant: XINJIANG TIANRUN BIOTECHNOLOGY CO., LTD., Urumqi (CN)

(72) Inventors: Xinling LI, Urumqi (CN); Guangqing MOU, DALIAN (CN); Xiaohong LUO, Urumqi (CN); Hong YOU, Urumqi (CN)

(73) Assignee: XINJIANG TIANRUN BIOTECHNOLOGY CO., LTD

(21) Appl. No.: 16/838,704

(22) Filed: Apr. 2, 2020

Related U.S. Application Data
(63) Continuation of application No. PCT/CN2018/097285, filed on Jul. 26, 2018.

(30) Foreign Application Priority Data
Mar. 7, 2018 (CN) 201810186371.1

(51) Int. Cl. C12C 12/00; C12C 12/04; A23C 9/12; C12N 1/16; C12N 1/18; C12R 1/645; C12R 1/865

(52) U.S. Cl. CPC C12C 12/006; C12C 12/04; A23C 9/1203; C12R 1/865; C12N 1/18; C12R 1/645; C12N 1/16

(57) ABSTRACT

The disclosure discloses a compound milk-beer lyophilized fermentation microorganism agent as well as a preparation and application thereof. The microorganism agent comprises a lyophilized microorganism agent of Kluyveromyces marxianus var. marxianus A3 and a lyophilized, microorganism agent of Saccharomyces cerevisiae TR2, wherein the ratio of the viable count of Kluyveromyces marxianus var. marxianus A3 to the viable count of Saccharomyces cerevisiae TR2 in the compound milk-beer lyophilized fermentation microorganism agent is (1.5-2.5):1. The compound fermentation microorganism agent can be applied in production of milk beer, and the product quality and production efficiency of milk beer are improved compared with single strains.

### CROSS-REFERENCE TO RELATED APPLICATIONS

This application is a continuation, of International Patent Application No. PCT/CN2018/097285 with a filing date of Jul. 26, 2018, designating the United States, now pending, and further claims priority to Chinese Patent Application No, 201810186371.1 with a filing date of Mar. 7, 2018. The content of the aforementioned applications, including any intervening amendments thereto, are incorporated herein by reference.

### TECHNICAL FIELD

The disclosure belongs to the field of biotechnology application, and particularly relates to a preparation method and application of a compound milk-beer fermentation microorganism agent.

### BACKGROUND OF THE PRESENT INVENTION

Milk beer is a novel fermented milk beverage with low alcohol volume number of molecules, features milky white and has strong malt fragrance, slightly carbonated, rich and white in foam, and fine and smooth in flavor; in addition, the milk beer also has the characteristics of sweet and sour taste and rich nutrition as milk beverage. The production technology of milk, beer was invented in China for the first time, and its suitable inoculants are lactic acid yeast and yeast.

At present, the research and application of yeast in China focus mostly on bread, wine and biopharmaceuticals, there are few researches on yeast for fermented dairy products, and there are almost no yeast, for milk beer. The applicant had previously disclosed a preparation method and application of a lyophilized microorganism agent of Kluyveromyces marxianus var. marxianus (CN107034149A) in which fermentation of milk beer using a single strain of Kluyveromyces marxianus var. marxianus A3 usually takes 16 hours, and is susceptible to the exterior. Once being influenced, the fermentation time can be prolonged even to more than 20 hours, and viscosity matter can generate to produce pollution so as to influence the quality. The applicant also found a strain of Saccharomyces cerevisiae TR2 used for producing milk beer, the ferementation speed is faster than that of Kluyveromyces marxianus var. marxianus A3, the acidity and the carbon dioxide content can meet the product requirements in only about 8 hours, and less likely for viscosity matter to generate and cause pollution. However, when Saccharomyces cerevisiae TR2 is used alone for milk beer fermentation, the alcohol content easily reaches the standard limit, which is 0.5%, specified by enterprises, at a risk of alcohol exceeding the standard. The two yeasts are universally recognized yeasts which are safe to environment and human beings, not to mention Saccharomyces cerevisiae as probiotic yeast, in which, case, the aforementioned defects can be avoided by compounding the two yeasts, and a mellower and richer flavor can be achieved after fermentation.

The research data show that the best flavor and quality are obtained when carbon dioxide content is 0.18-0.22%, alcohol content is 0.30-0.40% and acidity is 50-550 T according to the internal control standard. Although the prepared milk beer by using single strains of Kluyveromyces marxianus var. marxianus A3 and Saccharomyces cerevisiae TR2 can meet national food safety and enterprise standards and can be eaten safely, the optimal flavor may not be maintained in each batch of products. And the standard-reaching rate of the milk beer prepared from the single strains is not high enough, only 70-75%.

The applicant finds that after Kluyveromyces marxianus var. marxianus A3 and Saccharomyces cerevisiae TR2 are compounded, on one hand, the fermentation speed is faster than of that when in alone use by about 6 hours, and the flavor and the quality stability are superior to that when the single strain is used.

When the traditional liquid yeast strain is used for preparing the milk beer, the yeast strain needs to be continuously propagated for production, as such the problems of multiple preparation links, complex operation and potential pollution of strains occur, the strain quality is easy to fluctuate due to different strain preparation levels, consequently the quality of the milk beer product is caused to fluctuate, which is disadvantageous for quality control of large-scale milk beer production. After being subjected to vacuum freeze drying, the strain can maintain high physiological characteristics and biological activity, and can be directly put into food fermentation production.

In view of this, the disclosure provides a lyophilized fermentation microorganism agent compounded with Kluyveromyces marxianus var. marxianus A3 and Saccharomyces cerevisiae TR2 and application thereof.

### SUMMARY OF PRESENT INVENTION

An object of the disclosure is to provide a compound milk-beer lyophilized fermentation microorganism agent for producing milk beer. The lyophilized fermentation microorganism agent compounds Kluyveromyces marxianus var. marxianus A3 with Saccharomyces cerevisiae TR2, which not only renders a shorter fermentation period than that by using A3 alone, but also improves the product quality stability, so that the product quality and the production efficiency are improved, and the safety and the sanitation are further ensured.

In order to achieve the above object, the technical solution is adopted as follows:

Provided is a compound milk-beer lyophilized fermentation microorganism agent, comprising a lyophilized microorganism agent of Kluyveromyces marxianus var. marxianus and a lyophilized microorganism agent of Saccharomyces cerevisiae;

wherein the ratio of the viable count of Kluyveromyces marxianus var. marxianus to the viable count of Saccharomyces cerevisiae in the compound milk-beer lyophilized fermentation microorganism agent is (1.5-2.5):1;

the Kluyveromyces marxianus var. marxianus, with a strain number A3, has been conserved at China General Microbiological Culture Collection Center (CGMCC) on Apr. 27, 2010, with a conservation number CGMCC NO. 3781; and

the Saccharomyces cerevisiae, with a strain number TR2, has been conserved at China General Microbiological Culture Collection Center (CGMCC) on Dec. 4, 2017, with a conservation number CGMCC No. 14964.

Further, the ratio of the viable count of Kluyveromyces marxianus var. marxianus to the viable count of Saccharomyces cerevisiae in the compound milk-beer lyophilized fermentation microorganism agent is 2:1.

Further, the viable count of the lyophilized microorganism agent of Kluyveromyces marxianus var. marxianus is more than 2.0×108 cfu/g; and

the viable count of the lyophilized microorganism agent of Saccharomyces cerevisiae is more than 4.0×108 cfu/g.

Still further, the viable count of the lyophilized microorganism agent of Kluyveromyces marxianus var. marxianus is 2×108-3.2×108 cfu/g; and

the viable count of the lyophilized microorganism agent of Saccharomyces cerevisiae is 4×108-6.4×108 cfu/g.

Another object of the disclosure is to provide application of the above-mentioned compound milk-beer lyophilized fermentation microorganism agent. The application method is simple, convenient and easy to learn.

In order to achieve the object, the technical solution is adopted as follows:

The application of the compound milk-beer lyophilized fermentation microorganism agent in preparing fermented food.

Further, the fermented food is milk, beer.

Further, the compound milk-beer fermentation microorganism agent is directly added into a milk beer base material, fermented and cultured at 28° C., and the addition amount is 100 U/ton;

Or a liquid strain with the viable count of 4.0×108-6.0×108 cfu/mL is prepared from the compound milk-beer fermentation microorganism agent, inoculated into the milk beer base material at an inoculation amount of 3%, and fermented and cultured under 28° f.

Still further, the preparation of the liquid strain comprises the steps of:

(1) Preparing a Culture Medium:

uniformly mixing whole milk, glucose, potato starch, tomato concentrate juice, sodium citrate and water, and sterilizing to obtain a culture medium, wherein the culture medium comprises 30% of whole milk, 3% of glucose, 1% of potato starch, 0.5% of tomato concentrate juice, 0.05% of sodium citrate and the balance of purified water; and

(2) Culturing:

activating the compound milk-beer lyophilized fermentation microorganism agent according to claim 1, inoculating into the culture medium, and culturing for 8-10 hours at 26-30° C. to obtain the liquid strain.

Still further, the milk beer base comprises 30% of yogurt, 10% of white granulated sugar, 10% of fruit juice, 0.5% of a stabilizer, 0.1-0.14% of lactic acid, and the balance of purified water.

Still further, in the preparation of the liquid strain: the culture temperature is 28° C. and the viable count is 5.0×108 cfu/mL; and

in the milk beer base material, the content of lactic acid is 0.12%.

Compared with the prior art, the disclosure has the beneficial effects:

1. According to the preparation method and the application of the compound milk-beer lyophilized fermentation microorganism agent of the disclosure, the lyophilized fermentation microorganism agent compounds the lyophilized microorganism agent of Kluyveromyces marxianus var. marxianus A3 with the lyophilized microorganism agent of Saccharomyces cerevisiae TR2 in a ratio of their viable counts of (1.5-2.5):1; compared with the single strain of A3, the compound lyophilized fermentation microorganism agent can not only shorten the fermentation period by about 6 hours, but also improve the product quality stability, thus rendering a richer flavor, and the alcohol content is easily controlled to be lower than 0.5%, the qualified rate according to the internal control standard can reach more than 96%, so that the product quality and the production efficiency are improved, and safety and sanitation are further guaranteed. In addition, the preparation method is simple, easy to learn and convenient to use, and the prepared lyophilized microorganism powder can be used for fermenting dairy products, fermenting foods with low-alcohol, health-care foods, silage and other products.

2. According to the preparation method and the application of the compound milk-beer lyophilized fermentation microorganism agent of the disclosure, the lyophilized fermentation microorganism agent is adopted, as the result, not only is the culture volume decreased thus rendering a reduced occupation of apparatus, less apparatus investment, shortened production period and improved production efficiency, but also the application method is simple and easy to learn, production can be run successfully by personals trained a little even in lack of

professional technician, which is suitable for small and medium enterprises with low technical level and primitive production equipment.

3. According to the preparation method and the application of the compound milk-beer lyophilized fermentation microorganism agent of the disclosure, the lyophilized fermentation, microorganism agent comprises, a lyophilized microorganism agent of Kluyveromyces marxianus var, marxianus A3 and a lyophilized microorganism agent of Saccharomyces cerevisiae TR2, Kluyveromyces marxianus var. marxianus and Saccharomyces cerevisiae are recognized to be safe to environment and human beings, and Saccharomyces cerevisiae is allowed to be used for producing health-care foods as probiotics. Using the two strains as fermentation strains of the milk beer can improve the health-care function of the milk beer in addition to improving the production performance.

### DESCRIPTION OF THE DRAWINGS

FIG. 1 is a microscopic image of cells of Saccharomyces cerevisiae.

### DETAILED DESCRIPTION OF PREFERRED EMBODIMENTS

In order to further illustrate the preparation method and application of the compound milk-beer lyophilized fermentation microorganism agent provided by the disclosure to achieve the expected inventive object, the preparation method, application, embodiments, structures, characteristics and effects of the compound milk-beer lyophilized fermentation microorganism agent will be described in detail in combination with preferred examples. In the following description, the term “one example” or “an example” unnecessarily means the same example. Furthermore, the specific features, structures, or characteristics of one or more examples can be combined in any suitable form.

Before describing in detail the preparation method and application of the compound milk-beer lyophilized fermentation microorganism agent, it is necessary to further explain related, materials and the like mentioned in the disclosure so as to render a better effect.

The raw materials in the disclosure are all conventional products commercially available.

The Kluyveromyces marxianus var. marxianus mentioned in the disclosure, with a strain number A3, has been conserved at China General Microbiological Culture Collection Center (address: Institute of Microbiology, Chinese Academy of Sciences, No. 3, Yard 1, Beichenxi Road, Chaoyang District, Beijing) on Apr. 27, 2010, with a conservation number CGMCC NO. 3781; the morphological characteristics are as follows:

in a malt wort liquid culture medium: after three days of culture under 25 C, the cells are spherical, oval and elliptical, and (3.6-6.0)×(6.0-7.2) v m in size with the formation of precipitates;

in a malt wort agar slant culture medium: after one month of culture under 25° C., the mycelia are cheese-like, milky, smooth in surface, non-reflective and trimmed on edge; and

in a corn flour agar plate culture medium: no pseudohyphae is generated.

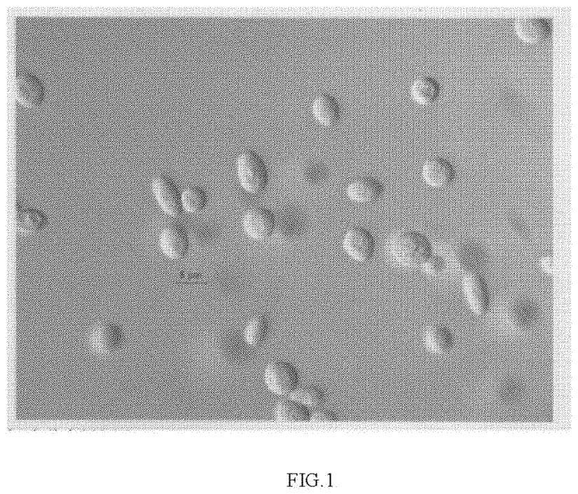

The Saccharomyces cerevisiae mentioned in the disclosure, with a strain number TR2, has been conserved at China General Microbiological Culture Collection Center (address: Institute of Microbiology, Chinese Academy of Sciences, No. 3, Yard 1, Beichenxi Road, Chaoyang District, Beijing) on Dec. 4, 2017, with a conservation number CGMCC No. 14964; the microscopic image of the cells is shown in FIG. 1.

U strain fermentation activity unit: the amount of bacteria capable of converting 1 micromole of substrate or the amount of bacteria, capable of converting 1 micromole of the relevant group in the substrate within 1 minute under the specified conditions (XC, otherwise the optimum conditions). Given the above relevant raw materials, the preparation method and the application of the compound milk-beer lyophilized fermentation microorganism agent are further described in detail in combination with specific Examples:

### Example 1

A. Preparation of a Lyophilized Microorganism Agent of Kluyveromyces Marxianus:

(1) preparing a seed liquid: streaking Kluyveromyces marxianus var. marxianus on a YEPD culture medium plate, placing in an incubator under 28° C. to culture for 1 day; picking out full strains, streaking again on the YEPD culture medium plate, placing in the incubator under 28° C. to culture for 1 day, inoculating the strains into a YEPD liquid culture medium, and culturing in a shaking flask under the conditions of 28° C. and 160 r/min overnight to obtain a seed solution;

(2) preparing a fermentation culture medium: weighing 67.37 g of sucrose, 29.7 g of yeast extract powder, 15.61 g of corn steep liquor, 4.13 g of monopotassium phosphate and 0.3 g of magnesium sulfate, dissolving in IL distilled water, uniformly mixing, and sterilizing at 121° C. for 20 min to obtain a fermentation medium;

(3) fermenting at high density: inoculating the seed solution into the fermentation culture medium, with an inoculation amount of 4×106 cfu/mL, under the conditions of 28° e and 160 r/min, culturing in a shaking flask for 16 hours and stopping the fermentation, thereby obtaining the high-density Kluyveromyces marxianus var. marxianus fermentation bacterial solution;

Preparation of a Lyophilized Microorganism Agent with a Vacuum Freeze Drying Method:

(4) centrifugal concentration: centrifuging the high-density Kluyveromyces marxianus var. marxianus fermentation bacterial solution for 15 min under the conditions of 4° C. and 6000 r/min, and pouring off supernatant to obtain Kluyveromyces marxianus var. marxianus active bacterial sludge;

(5) preparing a protective agent: preparing maltose with the concentration of 20 g/L, and sterilizing under 121° re for 20 min; and preparing skimmed milk powder with the concentration of 30 g/L,

sterilizing for 15 min under 115° C., cooling the prepared two protective agents, and mixing according to the volume ratio of 1:1 to obtain a composite protective agent;

(6) vacuum freeze drying: fully mixing Kluyveromyces marxianus var. marxianus active bacteria sludge prepared in step d with the composite protective agent obtained in step e in a volume ratio of 1:1, pre-freezing for 311 in a refrigerator under −80° C., and performing vacuum freeze drying for 24 h at a cryo-temperature of −40° t and a vacuum degree of 10 Pa to obtain the lyophilized microorganism agent of Kluyveromyces marxianus, wherein the viable count reached 3.2×108 cfu/g after vacuum freeze drying.

B. Preparation of a Lyophilized Microorganism Agent of Saccharomyces cerevisiae:

(1) Activating Strains:

streaking a slant of Saccharomyces cerevisiae stored in a 4° C. refrigerator in a YPD plate by inoculating ring, placing in a 28° C. incubator for 48 h, inoculating Saccharomyces cerevisiae with full colony morphology into a YPD liquid medium and culturing for 16 h in a shaking table under 28° C. and at a rpm of 150 r/min to obtain a seed solution;

(2) Preparing a Propagation Culture Medium:

weighing 53.8 g of glucose, 21.7 g of peptone, 10 g of yeast extract, 2.98 g of potassium dihydrogen phosphate, dissolving in 1 L distilled water, heating and stirring until the solution was clear with no insoluble material at the bottom, and transferring to an empty 3 L fermentor (BIOTECH-3JG) and sterilizing under high pressure at 121° (for 15 min;

(3) Fermenting at High Density:

inoculating the seed solution at a percentage of 7% into the propagation culture medium which was sterilized in step (2) and cooled to the normal temperature, culturing for 18 h under the conditions of the liquid loading amount being ⅓L, the aeration amount being 4 L/min, the dissolved oxygen being constant at 40%, the pH being constant at 5.0, the rpm being 550 r/min (dissolved oxygen interconnected), and the culture temperature being 28 C, in which case, in the obtained Saccharomyces cerevisiae fermentation liquor, the cell number of Saccharomyces cerevisiae reached 1.4×109 cfu/mL.

(4) Collecting Thalli:

centrifuging the Saccharomyces cerevisiae fermentation liquor for 10 min under the conditions of 4° C. and 7000 r/min, discarding supernatant, and collecting precipitates; centrifugally washing the precipitates twice with sterile physiological saline under 4° C. at a rpm of 7000 r/min for 10 min to obtain thalli;

(5) Preparing a Protective Agent:

taking skimmed reconstituted milk as a basic protective agent, supplementing 15 g of skimmed reconstituted milk, 2 g of sodium glutamate, 2 g of glycerol, 2 g of mannitol and 1 g of trehalose with distilled water to 100 g, uniformly mixing, and sterilizing for 15 min at 115° 15 to obtain the protective agent;

(6) Vacuum Freeze Drying:

adding 40 g of the protective agent prepared in step (2) into 20 g of the collected thalli, oscillating, uniformly mixing with a swirler, transferring to a lyophilization bottle, freezing for 3.5 h in a −85° b refrigerator, immediately transferring to a lyophilization machine which is pre-frozen and has a stable cryo-temperature, vacuumizing, and freeze-drying for 18 h, wherein the cryo-temperature was −60° t, the vacuum degree was 20 pa, in which case, the lyophilized microorganism agent of Saccharomyces cerevisiae was obtained, and the viable count could reach 6.4×108 cfu/g after lyophilization.

C. The lyophilized microorganism agents prepared in steps A and B, namely the lyophilized microorganism agent of Kluyveromyces marxianus var. marxianus A3 and the lyophilized microorganism agent of Saccharomyces cerevisiae TR2, were compounded according to the mass ratio of 4:1 to obtain the compound milk-beer lyophilized fermentation microorganism agent.

D. Application: The Compound Milk-Beer Lyophilized Fermentation Microorganism Agent was Directly Used for Producing the Milk Beer.

300 kg of yoghourt, 100 kg of white granulated sugar, 100 kg of fruit juice, 5 kg of a compound stabilizer and 1.2 kg of lactic acid were weighted, and supplemented with purified water to 1 ton of total weight, homogenized, sterilized, and cooled to 28° C. to obtain a milk beer base material;

addition was done according to the international specified activity unit of the lyophilized bacterial powder, 100 DCU of the compound-milk-beer lyophilized fermentation microorganism agent was added into 1 ton of milk-beer base material, then the compound milk-beer lyophilized fermentation microorganism agent was cultured for 10 hours at 28° C., the compound milk-beer lyophilized fermentation microorganism agent can be filled into a pop can or a glass bottle to be sterilized for 10 minutes under 80° y after being detected to have reached the specified index, and the compound lyophilized fermentation microorganism agent could be stored and put on sale at normal temperature after being sterilized.

Upon inspection, this batch of products were qualified.

After carrying out Example 1 for one year, the qualification rate, by the internal standard, of the product prepared throughout the year was 96.7%.

According to the preparation method and the application of the compound milk-beer lyophilized fermentation microorganism agent of the disclosure, the lyophilized fermentation microorganism agent compounds therein the lyophilized microorganism agent of Kluyveromyces marxianus var. marxianus A3 and the lyophilized microorganism agent of Saccharomyces cerevisiae TR2 at a ratio of their viable counts being 2:1; compared with the single strain of A3, the compound milk-beer lyophilized fermentation microorganism agent can not only shorten the fermentation period, but also improve the product quality stability, thus rendering a richer flavor, and the alcohol content is easily controlled to be lower than 0.5%, the qualified rate according to the internal control standard can reach more than 96%, so that the product quality and the production efficiency are improved, and safety and sanitation are further guaranteed. Moreover, by adopting the lyophilized fermentation microorganism agent, not only the culture volume is decreased thus rendering a reduced occupation of apparatus, less apparatus investment and shortened production period, but also the application method is simple and easy to learn, production can be run successfully by personals trained a little even in lack of professional technician, which is suitable for small and medium enterprises with low technical level and primitive production equipment.

### Example 2

A. Preparation of a Lyophilized Microorganism Agent of Kluyveromyces Marxianus:

In the (3) fermenting at high-density in step A of this example, the inoculation amount was 1×106 cfu/mL, other operation steps were the same as those in step A of Example 1, and the viable count of the prepared lyophilized microorganism agent of Kluyveromyces marxianus var. marxianus A3 reached 2.0×108 cfu/g.

B. Preparation of a Lyophilized Microorganism Agent of Saccharomyces cerevisiae:

In the (3) fermenting at high-density in step B of this example, the inoculation percentage was 3%, other operation steps were the same as those in step B of the example 1, and the viable count of the lyophilized microorganism agent of the prepared Saccharomyces cerevisiae TR2 reached 4.0×108 cfu/g.

C. The lyophilized microorganism agents prepared in steps A and B, namely the lyophilized microorganism agent of Kluyveromyces marxianus var. marxianus A3 and the lyophilized microorganism agent of Saccharomyces cerevisiae TR2, were compounded according to the mass ratio of 5:1 to obtain the compound milk-beer lyophilized fermentation microorganism agent.

D. Application:

(1) Preparation of a Culture Medium:

uniformly mixing whole milk, glucose, potato starch, tomato concentrate juice, sodium citrate (pH was adjusted to 5.00 with citric acid) and water, and carrying out sterilization under high pressure for 15 min at 115° C. to obtain a culture medium, wherein the culture medium comprises 30% of whole milk, 3% of glucose, 1% of potato starch, 0.5% of tomato concentrate juice, 0.05% of sodium citrate and the balance of water;

(2) Culturing:

activating the compound milk-beer lyophilized fermentation microorganism agent, inoculating into the culture medium, and culturing for 9 hours under 26° C. so that the viable count reached 5.0×108 cfu/mL, and a liquid strain was obtained;

(3) Preparation of a Milk Beer Base Material:

weighing 300 kg of yoghourt, 100 kg of white granulated sugar, 100 kg of fruit juice, 5 kg of a compound stabilizer and 1 kg of lactic acid, supplementing with purified water until the total weight was 1 ton, homogenizing, sterilizing at 95° C. for 5 min, sterilizing and cooling to 28° C. to obtain a milk beer base material; and

(4) the liquid strain was inoculated into the milk beer base material at an inoculation percentage of 3%, fermented and cultured for 9 hours at 28° C. the milk beer base material was filled into a pop can or a glass bottle after being detected to have reached a specified index, sterilized under 80° o for 10 minutes, and could be stored and put on sale at normal temperature after inactivation. Upon inspection, this hatch of products were qualified.

According, to the preparation method and the application of the compound milk-beer lyophilized fermentation microorganism agent of the disclosure, the lyophilized fermentation, microorganism agent compounds the lyophilized microorganism agent of Kluyveromyces marxianus var. marxianus A3 with the lyophilized microorganism agent of Saccharomyces cerevisiae TR2 at a ratio of their viable counts being 2.5:1; compared with the single strain of A3, the compound milk-beer lyophilized fermentation microorganism agent can not only shorten the fermentation period, but also improve the product quality stability, thus rendering a richer flavor, and the alcohol content is easily controlled to be lower than 0.5%, the qualified rate according to the internal control standard can reach more than 96%, so that the product quality and the production efficiency are improved, and safety and sanitation are further guaranteed. Moreover, by adopting the lyophilized fermentation microorganism agent, not only the culture volume is decreased thus rendering a reduced occupation of apparatus, less apparatus investment and shortened production period, but also the application method is simple and easy to learn, production can be run successfully by personals trained a little even in lack of professional technician, which is suitable for small and medium enterprises with low technical level and primitive production equipment.

### Example 3

A. Preparation of a Lyophilized Microorganism Agent of Kluyveromyces Marxianus:

in the (3) fermenting at high density in step A of this example, the inoculation amount was 3×106 cfu/mL, other operation steps were the same as those in step A of Example 1, and the viable, count of the prepared lyophilized microorganism agent of Kluyveromyces marxianus var. marxianus A3 reached 2.5×108 cfu/g.

B. Preparation of a Lyophilized Microorganism Agent of Saccharomyces cerevisiae:

in the (3) fermenting at high density in step B of this example, the inoculation percentage was 4%, other operation steps were the same as those in step B of Example 1, and the viable count of the lyophilized microorganism agent of the prepared Saccharomyces cerevisiae TR2 reached 5.0×108 cfu/g.

C. The lyophilized microorganism agents prepared in steps A and B, namely the lyophilized microorganism agent of Kluyveromyces marxianus var. marxianus A3 and the lyophilized microorganism agent of Saccharomyces cerevisiae were compounded according to the mass ratio of 3:1 to obtain the compound milk-beer lyophilized fermentation microorganism agent.

D. Application:

(1) Preparation of a Culture Medium:

uniformly mixing whole milk, glucose, potato starch, tomato concentrate juice, sodium citrate (pH, was adjusted to 5.00 by citric acid) and water, and carrying out sterilization under high pressure for 15 min at 115° d to obtain a culture medium, wherein the culture medium comprises 30% of whole milk, 3% of glucose, 1% of potato starch, 0.5% of tomato concentrate juice, 0.05% of sodium citrate and the balance of water;

(2) Culturing:

activating the compound milk-beer lyophilized fermentation microorganism agent, inoculating into the culture medium, culturing for 8 hours under 30° C. so that the viable count reached 4.0×108 cfu/mL, and a liquid strain was obtained;

(3) Preparation of a Milk Beer Base Material:

weighing 300 kg of yoghourt, 100 kg of white granulated sugar, 100 kg of fruit juice, 5 kg of a compound stabilizer, 1.4 kg of lactic acid and the balance of purified water until the total weight was 1 ton, homogenizing, sterilizing at 95V, for 5 min, sterilizing and cooling to 28° C. to obtain a milk beer base material; and

(4) the liquid strain was inoculated into the milk beer base material at an inoculation percentage of 3%, fermented and cultured for about 10 hours under 28° C., the milk beer base material was filled into a pop can or a glass bottle after being detected to have reached a specified index, sterilized under 80° for 10 minutes, and could be stored and put on sale at normal temperature after sterilization.

Upon inspection, this batch of products were qualified according to the internal standard.

According to the preparation method and the application of the compound milk-beer lyophilized fermentation microorganism agent of the disclosure, the lyophilized fermentation microorganism agent compounds the lyophilized microorganism agent of

Kluyveromyces marxianus var. marxianus A3 with the lyophilized microorganism agent of Saccharomyces cerevisiae TR2 at a ratio of their viable counts being 1.5:1; compared with the single strain, of A3, the compound milk-beer lyophilized fermentation microorganism agent can not only shorten the fermentation period, but also improve the product quality stability, thus rendering a richer flavor, and the alcohol content is easily controlled to be lower than 0.5%, the qualified rate according to the internal control standard can reach more than 96%, so that the product quality and the production efficiency are improved, and safety and sanitation are further guaranteed. Moreover, by adopting the lyophilized fermentation microorganism agent, not only the culture volume is decreased thus rendering a reduced occupation of apparatus, less apparatus investment and shortened production period, but the application method is simple and easy to learn, production can be run successfully by personals trained a little even in lack of professional technician, which is suitable for small and medium enterprises with low technical level and primitive production equipment.

### Example 4

A. Compound Milk-Beer Lyophilized Fermentation Microorganism Agent

The operation steps for preparing the compound milk-beer fermentation microorganism agent in this example, were the same as those in Example 1.

B. Application:

(1) Preparation of a Culture Medium:

uniformly mixing whole milk, glucose, potato starch, tomato concentrate juice, sodium citrate (pH was adjusted to 5.00 with citric acid) and water, and carrying out sterilization under high pressure for 15 min at 115° C. to obtain a culture medium, wherein the culture medium comprises 30% of whole milk, 3% of glucose, 1% of potato starch, 0.5% of tomato concentrate juice, 0.05% of sodium citrate and, the balance of water;

(2) Culturing:

activating the compound milk-beer lyophilized fermentation microorganism agent for milk beer, inoculating into the culture medium, culturing for 10 hours under 28° C., wherein the viable count reached 6.0×108 cfu/mL, and obtaining a liquid strain:

(3) Preparation of a Milk Beer Base Material, Including:

weighing 300 kg of yoghourt, 100 kg of white granulated sugar, 100 kg of fruit juice, 5 kg of a compound stabilizer and 1.2 kg of lactic acid were weighed, supplementing with purified water until the total weight was 1 ton, homogenizing, sterilizing at 95 C for 5 min, sterilizing at 96 C for 25 min in a strain tank, sterilizing and cooling to 28 C to obtain a milk beer base material; and

(4) the liquid strain was inoculated into the milk beer base material at an inoculation amount of 3%, fermented and cultured for 8 hours under 28° C., the milk beer base material was filled into a pop can or a glass bottle after being detected to have reached a specified index, sterilized under 80° for 10 minutes, and could be stored and put on sale at normal temperature after sterilization.

Upon inspection, this batch of products were qualified according to the internal standard.

After carrying out Example 4 for one year, the qualification rate, by the internal standard, of the product prepared throughout the year was 97%.

According, to the preparation method and the application of the compound milk-beer lyophilized fermentation microorganism agent of the disclosure, the lyophilized fermentation microorganism agent compounds the lyophilized microorganism agent of Kluyveromyces marxianus var. marxianus A3 with the lyophilized microorganism agent of Saccharomyces cerevisiae TR2 at a ratio of their viable counts being 2:1; compared with the single strain of A3, the compound milk-beer lyophilized fermentation microorganism agent can not only shorten the fermentation period, but also improve the product quality stability, thus rendering a richer flavor, and the alcohol content is easily controlled to be lower than 0.5%, the qualified rate according to the internal control standard can reach more than 96%, so that the product quality and the production efficiency are improved, and safety and sanitation are further guaranteed. Moreover, by adopting the lyophilized fermentation microorganism agent, not only the culture volume is decreased thus rendering a reduced occupation of apparatus, less apparatus investment and shortened production period, but the application method is simple and easy to learn, production can be run successfully by personals trained a little even in lack of professional technician, which is suitable for small and medium enterprises with low technical level and primitive production equipment.

The above is only preferred examples of the disclosure, and is not intended to limit the examples of the disclosure. Any simple modifications, equivalent changes and alternations made to the above examples in accordance with the technical spirit of the examples of the disclosure are still within the scope of the technical solutions of the embodiment of the disclosure.

We claim:

1. A compound milk-beer lyophilized fermentation microorganism, agent, wherein,
the compound milk-beer fermentation microorganism agent comprises a lyophilized microorganism agent of Kluyveromyces marxianus var. marxianus and a lyophilized microorganism agent of Saccharomyces cerevisiae;
wherein, the ratio of the viable count of Kluyveromyces marxianus var. marxianus to the viable count of Saccharomyces cerevisiae in the compound milk-beer lyophilized fermentation microorganism agent for milk beer is (1.5-2.5):1;
the Kluyveromyces marxianus var. marxianus, with a strain number A3, has been conserved at China General Microbiological Culture Collection Center (CGMCC) on Apr. 27, 2010, with a conservation number CGMCC NO. 3781; and
the Saccharomyces cerevisiae, with a strain number TR2, has been conserved at China General Microbiological Culture Collection Center (CGMCC) on Dec. 4, 2017, with a conservation number CGMCC No. 14964.

2. The compound milk-beer lyophilized fermentation microorganism agent according to claim 1, wherein,
the ratio of the viable count of Kluyveromyces marxianus var. marxianus to the viable count of Saccharomyces cerevisiae in the compound milk-beer lyophilized fermentation microorganism agent is 2:1.

3. The compound milk-beer lyophilized fermentation microorganism agent according to claim 1, wherein,
the viable count of the lyophilized microorganism agent of Kluyveromyces marxianus var. marxianus is more than 2.0×108 cfu/g; and
the viable count of the lyophilized microorganism agent of Saccharomyces cerevisiae is more than 4.0×108 cfu/g.

4. The compound milk-beer lyophilized fermentation microorganism agent according to claim 3, wherein,
the viable count of the lyophilized microorganism agent of Kluyveromyces marxianus var. marxianus is 2×108-3.2×108 cfu/g; and
the viable count of the lyophilized microorganism agent of Saccharomyces cerevisiae is 4×108-6.4×108 cfu/g.

5. Application of the compound milk-beer lyophilized fermentation microorganism agent according to claim 1 in preparation of fermented food.

6. The application according to claim 5, wherein,
the fermented food is milk beer.

7. The application according to claim 6, wherein,
the compound milk-beer fermentation microorganism agent according to claim 1 is directly added into a milk beer base material, fermented and cultured at 28° 8, with addition amount being 100 U/ton;
or a liquid strain with the viable count of 4.0×108-6.0×108 cfu/mL is prepared from the compound milk-beer fermentation microorganism agent according to claim 1, inoculated into the milk beer base material in an inoculation amount of 3%, and fermented and cultured at 28° 8 d.

8. The application according to claim 7, wherein,
the preparation method of the liquid strain comprises the steps of:
(1) preparing, a culture medium:
uniformly mixing whole milk, glucose, potato starch, tomato concentrated juice, sodium citrate and water, and sterilizing to obtain a culture medium, wherein the culture medium comprises 30% of whole milk, 3% of glucose, 1% of potato starch, 0.5% of tomato concentrate juice, 0.05% of sodium citrate and the balance of purified water; and
(2) culturing:
activating the compound milk-beer lyophilized fermentation microorganism agent according to claim 1, inoculating into the culture medium, and culturing for 8-10 hours at 26-30° 6 to obtain the liquid strain.

9. The application according to claim 8, wherein,
the milk beer base comprises the following components: 30% of, yogurt, 10% of white granulated sugar, 10% of fruit juice, 0.5% of a stabilizer, 0.1-0.14% of lactic acid, and the balance of purified water.

10. The application according to claim 9, wherein,
in the preparation of the liquid strain: the culture temperature is 28° C. and the viable count is 5.0×108 cfu/mL; and
in the milk beer base material, the content of lactic acid is 0.12%.
